(19) United States
(12) Patent Application Publication
(10) Pub. No.: US 2007/0244305 A1
(43) Pub. Date: Oct. 18, 2007

(54) Process for The Manufacture of Virus Safe Immunoglobulin

(75) Inventor: Jaakko Parkkinen, Espoo (FI)

Correspondence Address: SUGHRUE MION, PLLC, 2100 PENNSYLVANIA AVENUE, N.W., SUITE 800, WASHINGTON, DC, 20037, US

(73) Assignee: Suomen Punainen Risti Veripalvelu, Helsinki (FI)

(21) Appl. No.: 10/585,745

(22) PCT Filed: Jan. 31, 2005
(86) PCT No.: PCT/FI05/00064

§ 371 Date: Feb. 5, 2007

Related U.S. Application Data
(60) Provisional application No. 60/539,999, filed on Jan. 30, 2004.

(51) Int. Cl. C07K 1/34

(52) U.S. Cl. 530/390.1; 530/414

(57) ABSTRACT

Process for preparing a purified immunoglobulin preparation. The process comprises the steps of subjecting a crude immunoglobulin solution to caprylic acid treatment, removing protein aggregates and viruses from the immunoglobulin solution, subjecting the immunoglobulin solution to anion exchange chromatography in order to purify the immunoglobulin, filtering the immunoglobulin solution thus obtained on a virus-removal filter to produce an eluate containing immunoglobulin, and recovering the immunoglobulin. By combining caprylic acid treatment and precipitation with a protein precipitant the level of aggregated proteins and viruses is effectively reduced and a truly virus safe preparation is provided after filtration.

### BACKGROUND OF THE INVENTION

1. Field of the Invention

The present invention relates to the production of immunoglobulins. In particular, the present invention concerns a process for manufacturing a virus safe immunoglobulin composition, which is suitable for, e.g., parenteral administration. The invention also concerns novel virus safe immunoglobulin compositions and a method of purifying immunoglobulin solutions by nanofiltration.

2. Description of Related Art

Immunoglobulins, also called antibodies, can be extracted from blood plasma and they can be produced by hybridoma technology and recombinant DNA technology. In view of their broad scope of biological activity, antibodies are valuable therapeutic agents.

Immunoglobulin purified from normal human plasma has proved effective in the treatment of various serious diseases when administered intravenously. The pharmaceutical product is called “intravenous immunoglobulin” (Immune Globulin Intravenous Human or Human Normal Immunoglobulin for Intravenous Administration), which in the following appears in its vernacular abbreviated form “IVIG”. Due to the large intravenous doses administered, safety and tolerability of IVIG products are a specific concern.

Serious adverse effects caused by IVIG products have been ascribed to immunoglobulin aggregates, to other contaminating proteins and to blood-borne viruses. Immunoglobulin polymers and aggregates activate complement and their removal from IVIG products is considered important. The introduction of screening of donated blood and plasma for viral markers and implementation of effective virus inactivation methods has greatly improved the safety of the current IVIG products. However, a risk of viral transmission still exists, particularly with physico-chemically resistant viruses, such as parvovirus B19, which is not effectively inactivated by current chemical virus inactivation methods (Knezevic-Maramica and Kruskall, Transfusion 43, 1460-1480, 2003).

Immunoglobulin has traditionally been prepared from human plasma by the cold ethanol fractionation method according to Cohn and Oncley (Oncley et al., J Am Chem Soc, 71, 541-550, 1949) and its subsequent modifications. Such immunoglobulin preparations can only be administered subcutaneously or intramuscularly because of adverse effects associated with their intravenous infusion. Therefore, other manufacturing steps have been added by individual manufacturers for removal of aggregates and other contaminants and inactivation of viruses. However, the addition of multiples steps to manufacturing lowers the yield of immunoglobulin and raises the manufacturing costs. At the same time, the increasing demand of IVIG products has made the yield a critical issue.

The chemical virus inactivation methods currently used are effective against lipid-enveloped viruses but do not—as already indicated above—inactivate non-enveloped viruses, such as parvovirus and hepatitis A virus. Considering the potential load of physico-chemically resistant viruses such as parvovirus B19 in plasma pools (Schmidt et al., Vox Sang. 81, 228-235, 2001), the manufacturing process should be able to reduce a very high amount of non-enveloped viruses in order to yield a truly virus-free product. The current manufacturing processes do not meet this requirement and IVIG products may transmit parvovirus (Hayakaxa et al., Br J Haematol. 118, 1187-1189, 2002).

In the art, there are some known processes for producing purified intravenous immuno-globulin. Thus, U.S. Pat. Nos. 5,886,154 and 6,307,028 disclose a high-yield process for manufacturing a purified, virally inactivated antibody preparation from a starting solution. However, the process has only limited capacity to remove physico-chemically resistant infectious agents.

Published International Patent Application WO 99/64462 relates to a process for purifying immunoglobulin G from a crude immunoglobulin-containing plasma protein fraction. The known process includes the steps of anion exchange chromatography and cation exchange chromatography connected twice in series. Before chromatography, a protein precipitant is added to the immunoglobulin suspension. Virus-inactivation is performed by an S/D treatment.

The yield of this known process is only moderate because rather high concentration of protein precipitant and four chromatographic steps are used, the latter because the immunoglobulin has to be separated from the S/D-treated solution.

Both the above processes include steps of virus inactivation, but the process steps are not sufficient to provide for efficient virus removal, which would yield a product that could be characterized as being virus safe. Furthermore, generally, the more effective a virus-removal step is, the lower the overall yield of the process.

Nanofiltration (virus-removal filtration) provides an efficient means for removing non-enveloped viruses from solutions of biologically active proteins. In our U.S. Pat. Nos. 6,251,860 and 6,326,473 we describe a process for producing virus-safe apotransferrin using, i.a., a nanofiltration step using a filter having an average pore size in the range of 10 to 30 nm. However, we have found that solutions of immunoglobulins are difficult to filter with a nanofilter because the filter becomes rapidly clogged.

### SUMMARY OF THE INVENTION

It is an aim of the present invention to eliminate at least some of the above mentioned problems of the art and to provide a novel high-yield manufacturing process of immunoglobulin, which makes it possible to manufacture aggregate-free and virus-free immunoglobulin.

It is another object of the invention to provide novel virus-safe immunoglobulin compositions.

It is still a third object of the invention to provide a novel method of purifying immunoglobulin solutions by nanofiltration.

The present invention is based on the finding that it is possible effectively to precipitate aggregated proteins and viruses while retaining monomeric immunoglobulin in solution when the process includes a treatment step with a low amount of protein precipitant or adsorbent suitably carried out in conjunction with another treatment step, in which the solution is contacted with caprylic acid. Due to the combined effect of the protein precipitant or adsorbent step and of the caprylic acid treatment, an effective removal of viruses is, viz., obtained. If, for example, a protein precipitant, such as polyethylene glycol, would be used alone, a higher concentration would be needed for effective virus removal, which decreases the yield of immunoglobulin.

Based on the above, a novel manufacturing process has been designed, which comprises the steps of
A. subjecting a crude immunoglobulin solution to caprylic acid treatment;
B. removing protein aggregates and viruses from the immunoglobulin solution by treatment with a precipitant or adsorbent;
C. purifying immunoglobulin by anion exchange chromatography; and
D. filtering aggregate-free immunoglobulin solution with a virus-removal filter.

Steps B and C can be carried out in optional order; they are, however, carried out after step A and before step D.

As a result of the above processing steps, a virus-safe immunoglobulin solution is obtained which contains no detectable protein polymers or aggregates. It can be converted into an immunoglobulin composition, which contains high concentrations of immunoglobulin (up to 250 g/l of immunoglobulin). Furthermore, surprisingly, treatment steps A to C will result in an immunoglobulin solution, which can be filtered with high immunoglobulin throughput on a virus-removal filter while maintaining a high flux throughout the filtering operation.

More specifically, the process according to the present invention is mainly characterized by what is stated in the characterizing parts of claims 1 and 6, respectively.

The immunoglobulin composition is characterized by what is stated in the characterizing part of claim 17, and the filtering method by what is stated in the characterizing part of claim 18.

Considerable advantages are obtained by the present invention. Thus, the process according to the present invention provides for the manufacture of virus-free immunoglobulin solution with high yield from human plasma. This is based on the combination of a virus inactivation step performed with caprylic acid and two virus removal steps, which both effectively remove even physico-chemically resistant viruses. The first virus removal step also removes aggregated proteins and thereby makes it possible to carry out the second virus removal step, virus filtration, with high yield and filtration capacity. The novel manufacturing process has an exceptionally large capacity to remove physico-chemically resistant viruses and other infectious agents, such as prions. The combination of these two virus removal steps gives unexpected benefits.

The implementation of several steps, which effectively remove even physico-chemically resistant viruses forms a major difference between the process of the present invention and the process according to U.S. Pat. Nos. 5,886,154 and 6,307,028.

In the following, the invention will be examined more closely with the aid of a detailed description and with reference to some working examples.

### BRIEF DESCRIPTION OF THE DRAWINGS

FIG. 1 shows the flow chart of a preferred embodiment of the present process.

### DETAILED DESCRIPTION OF THE INVENTION

Within the scope of the present invention, the term “immunoglobulin” designates monoclonal and polyclonal immunoglobulins selected from the group of IgG and IgA. In the following description, the invention is described in more detail using human polyclonal IgG as an example. However, it should be noted that the invention is applicable to other polyclonal and monoclonal antibodies suitably modified, if necessary, to take into account the different sources and therapeutic use of the immunoglobulin.

The present method can be applied to produce virus-safe immunoglobulin from other immunoglobulin sources than plasma, such as animal cell cultures and transgenic animals.

As explained above, the present invention generally comprises four essential steps, whereby in the first process step a crude immunoglobulin solution is subjected to caprylic acid treatment carried out at a pH below 5, preferably at 4.0 to 5.0. The caprylic acid treatment results in the inactivation of enveloped viruses. After caprylic acid treatment, the supernatant solution contains potential non-enveloped viruses and some protein aggregates, which are derived from the starting material and first processing steps. They are removed using a protein precipitant, such as polyethylene glycol (PEG), or adsorbent, such as fumed silica, at a pH in excess of 5.0. The pH of the supernatant solution is raised to 5.3 or higher, polyethylene glycol is added and the formed precipitate is removed by filtration. Alternatively, the supernatant solution is treated with fumed silica, which is removed by filtration. Final purification of immunoglobulin is accomplished by passing the solution through a column of anion exchange chromatography gel. The aggregate-free immuno-globulin solution is subjected to virus filtration. The virus-free immunoglobulin solution is concentrated and diafiltered by ultrafiltration. The resulting immunoglobulin solution is stable after formulation as a liquid formulation or as a freeze-dried powder.

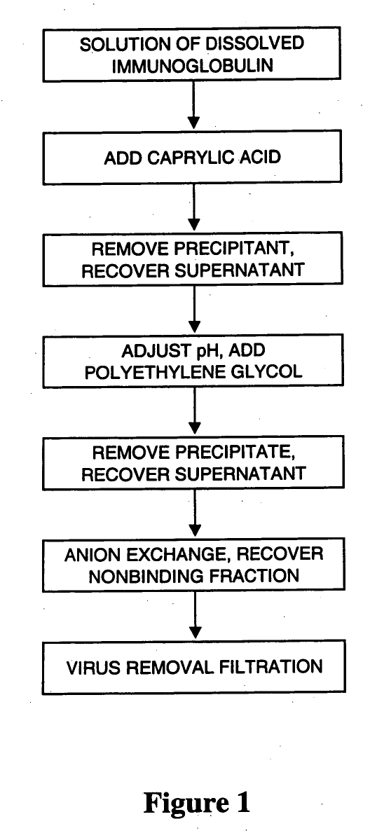

These main process steps are also shown in the embodiment of FIG. 1. Thus, the process begins by dissolving precipitated immunoglobulin, such as Cohn fraction II+III paste, in purified water or aqueous buffer. For filter aid-free fraction II+III paste, it is practical to use 8 volumes (w/v) of water although larger volumes may be also used. The pH of the suspension is adjusted below 5, preferably to about 4.8 (e.g. with 0.2 mol/l acetic acid). The suspension is mixed at about 5° C. until immunoglobulin has dissolved and the solution is warmed to 20-25° C. Caprylic acid is added slowly to a final concentration of 15-60 mmol/l, preferably to 20-50 mmol/l. During the treatment, the suspension is mixed. According to one preferred embodiment, the total amount of caprylic acid (or corresponding caprylate salt), determined based on virus inactivation evaluation, is added over a time period of about 15 minutes to 2 hours. Total treatment time with caprylic acid is from 15 minutes to 4 hours. Longer incubation times, e.g. up to about 16 hours, may be used but this leads to somewhat lower yield. Precipitated proteins and lipids are removed by centrifugation or filtration.

For the removal of aggregated proteins and viruses, the pH of the solution is raised to 5.3 or higher, preferably to about 5.4. Polyethylene glycol is added to a concentration of 10-50 g/l, preferably 20-40 g/l, in particular about 30 g/l. The use of higher PEG concentrations decreases immunoglobulin yield while the use of lower PEG levels decreases virus and aggregate removal. The molecular weight of the PEG is generally within the range of 3000 to 8000 Da, 3350 to 6000 Da being particularly preferred. In the examples below, PEG 4000 has been used. Treatment time with PEG may vary depending on the concentration of the PEG and the quality of the starting material. Typically, the duration of the PEG treatment is in the range of from 30 min to 20 hours, but shorter or even longer times of up to, e.g., 36 hours are possible.

A filter aid (for example, 20 g/l of diatomaceous earth) is added and the suspension is filtered. The clarified solution is passed through an anion exchange chromatography column. The anion exchange resin and chromatography conditions are chosen for their ability to selectively remove protein impurities, while maintaining high yield and subclass composition of IgG. Satisfactory purification is obtained, for example, by using an ANX Sepharose FF gel. The flow-through of the column containing IgG is collected. IgA is retarded by the column and can be eluted by increasing conductivity of the elution buffer with sodium chloride. The pH of the effluent solution is adjusted preferably to about 4.2-5.0.

The anion exchange resin may be based on various materials with respect to the matrix as well as to the attached charged groups. For example, the following matrices may be used, in which the materials mentioned may be more or less crosslinked: agarose based, cellulose based, dextran based, silica based and synthetic polymer based. The charged groups which are covalently attached to the matrix may e.g. be diethylaminopropyl (ANX), diethylaminoethyl (DEAE), quaternary aminoethyl (QAE), and/or quaternary ammonium (Q). Two or more anion exchange resins may be combined.

The aggregate-free immunoglobulin solution thus obtained is filtered with a virus-removal filter. Preferably, a filter capable of effectively removing even small non-enveloped viruses, such parvoviruses, is used. As used herein, to “effectively remove viruses” means to reduce the virus titre by at least about 3 log units and most preferably by about 4 log units or more. “Aggregate-free solution” refers herein to a solution without detectable protein polymers or aggregates in size exclusion chromatography.

The virus-filtered solution is concentrated by ultrafiltration. The level of polyethylene glycol is lowered by diafiltration so that its concentration in the final immunoglobulin solution is below 2 g/l. The ultrafiltration removes most of the polyethylene glycol, although there will remain some PEG depending on the concentration of immunoglobulin in the effluent of the ultrafiltration. For an IVIG product, the pH is adjusted to 3.8 to 5.8 and osmolarity is adjusted, for example, with glycine, other amino acids, sugars or polyols to be compatible for intravenous injection. The final solution containing 5-20% IgG is sterile filtered and dispensed into final containers. The product is stable as a liquid formulation but may also be freeze dried. The purity of immunoglobulin in the final product is at least 98% as analyzed by zone electrophoresis and does not contain detectable polymers or aggregates in size exclusion chromatography. The detection limit in size exclusion chromatography is about 0.1 wt. %.

The unexpected benefit of combining caprylic acid treatment and precipitation with a protein precipitant is that aggregated proteins and viruses are effectively reduced. The level of aggregated proteins is reduced to the extent that after purification with anion exchange chromatography, the immunoglobulin solution can be filtered with high capacity through a small pore size virus filter. Removal of aggregates is a prerequisite for the efficient filtration of immunoglobulin solution through such a virus filter because protein aggregates clog filter pores, which results in decreasing flux and impairs virus removal (Hirasaki et al., Membrane 20, 135-142, 1995). To date, efficacious filtration of intact immuno-globulins through a small pore size virus removal filter has not been possible. “Small pore size” means herein that the filter membrane removes at least 3 log of viruses with a particle size of about 20 nm, such as parvovirus. “Efficacious filtration” means herein that at least 5 kg of immunoglobulin can be passed through 1 m² of filter area with less than 50% decrease in filter flux. This enables cost-effective industrial-scale virus filtration. Virus filtration with a small pore size virus-removal filter is an advantageous manufacturing step, as it removes not only small non-enveloped viruses, but also other physico-chemically resistant infectious agents, such as prions.

The importance of the specific aggregate removal step for successful performance of virus filtration is illustrated in Example 3. By combining the polyethylene glycol precipitation step with virus filtration, the present invention comprises two effective steps for the removal of resistant non-enveloped viruses, which is illustrated in Example 2.

Based on the above, the present invention also concerns a method of efficaciously filtering immunoglobulin solutions on a nanofilter having a pore size of 10 to 40 nm, preferably about 10 to 30 nm, which comprises conducting through the filter a pure immunoglobulin solution, which contains about 2 to 4 wt.-% polyethylene glycol and no detectable polymer aggregates to remove at least 3 log of viruses with particle size of about 20 nm. More than 5 kg, preferably at least 7.5 kg, of immunoglobulin can be passed through 1 m² of filter area with a decrease of less than 70%, in particular less than about 50%, in filter flux.

For the purpose of the present invention, the average pore size of a virus-removal filter can be calculated on the basis of water flow by the Hagen-Poiseulle equation, which assumes a uniform distribution of equally-sized cylindrical pores.

$$\begin{matrix}{D}_{a ∀} = 2.0 \times {( Jd η \text{/} Δ P α )}^{\frac{1}{2}} \\ where , \end{matrix}$$

$$\begin{matrix} {D}_{α ∀} [ nm ] & mean pore diameter \\ J [ ml \text{/} min \text{/} {m}^{2} ] & water flow rate ( flux ) \\ d [ μ M ] & wall thickness \\ η [ centipoise ] & viscosity of water \\ Δ P [ mm Hg ] & filtration pressure \\ α [ - ] & porosity\end{matrix}$$

Basically, any virus-removal filter having the indicated pore size or providing a corresponding virus-removal efficiency can be utilized. We have found that particularly good results are obtained by using a composite filter of the kind disclosed in U.S. Pat. No. 5,096,637, the contents of which is herewith incorporated by reference. Such filters typically exhibit an asymmetric composite membrane structure. They comprise a skin layer with ultrafiltration properties, a porous substrate and a porous intermediate zone between the skin layer and the substrate.

Filters which can be used in virus filtration according to the present invention include, but are not limited to, Viresolve NFP (Millipore), Planova 15N and 20N (Asahi Kasei) and DV20 (Pall).

The aqueous solution subjected to virus-removal filtration preferably contains about 1 to 25 g/l of immunoglobulin, such as IgG or IgA. The immunoglobulin solution has a purity of more than 95%, preferably more than 98%. The pH of the solution is preferably in the range of 4.2 to 5.0 in particular about 4.2 to 4.8. Unexpectedly, it was found that filtrate flux and immunoglobulin throughput were clearly increased when pH of the immunoglobulin solution was lowered from 5.2 to 4.4, but decreased again at pH 4.2. The preferred range will vary within the broad range of 4.2 and 5.0 depending on the immunoglobulin, which is subjected to filtration.

Filtering is preferably carried out at a temperature of 20 to 50° C. and at a transmembrane pressure of about 0.2 to 8 bar, preferably of about 0.5 to 5.5 bar. In order to remove any particles that might be present, it is possible to prefilter the immunoglobulin solution though a filter having an average pore size of about 0.05 to 0.2 μm. It is preferred to carry out the nanofiltration at elevated temperature, such as 30 to 40° C., because this will increase flux. An elevated temperature at low pH (about 4.2 to 4.6) will be beneficial not only for the performance of the virus filtration but also for reduction of any anti-complementary activity of the immunoglobulin solution, which is a pharmacopoieal requirement of IVIG compositions.

Although filtering is preferably carried out for an immunoglobulin solution obtained by processing steps A to C described above, the present filtering method is generally applicable to any immunoglobulin solution, which contains polyethylene glycol or has been treated with an adsorbent and which—in a similar way—is free from detectable amounts of protein aggregates. Thus, it is possible to modify for example the known immunoglobulin processes of U.S. Pat. Nos. 5,886,154 and 6,307,028 by adding polyethylene glycol to the immunoglobulin solutions produced thereby at a suitable stage of the process to remove protein aggregates, and then to subject the final immunoglobulin solution to virus-removal filtration.

U.S. Pat. Nos. 5,886,154 and 6,307,028 describe a purification method for antibodies (immunoglobulin) using caprylic acid precipitation and chromatography. In the present invention, caprylic acid precipitation is preferably carried out by adding caprylic acid as a free acid instead of adding it in the form of a salt, such as sodium caprylate, as in U.S. Pat. Nos. 5,886,154 and 6,307,028. Caprylic acid slightly lowers the pH of the solution in contrast to sodium caprylate, which increases the pH. In the present invention, no pH shift to pH 5.0-5.2 according to U.S. Pat. Nos. 5,886,154 and 6,307,028 takes place and virus inactivation is carried out at a lower pH. This is beneficial since at low pH the proportion of the non-ionized form of caprylic acid is higher, and it is the non-ionized form of caprylic acid, which is effective in virus inactivation. However, allowing for the above disadvantages, “caprylic acid treatment” can also be carried out in the present invention using a caprylate salt as taught by the cited US Patents and under the conditions mentioned therein.

As discussed above, parvovirus B19 remains a risk with current plasma products as plasma pools may contain up 10¹⁰ genome equivalents of parvovirus or more per ml and it is resistant to chemical virus inactivation procedures. In the manufacturing process according to the present invention, it is preferred to screen the starting plasma by PCR for parvovirus B19 DNA, and to exclude plasma units containing ≧104 IU/ml from manufacturing. The international unit (IU) refers herein to the WHO International Standard for parvovirus B19 DNA. As the smallest infectious dose of parvovirus, which is capable of transmitting infection through intravenous infusion, is not known, the manufacturing process should remove all parvovirus potentially present in the starting plasma pool. By applying the cut-off level of 10⁴ IU/ml in the screening, the highest potential load of parvovirus in industrial plasma pools containing several thousand liters will be in the order of 10¹⁰-10¹¹ IU. Because the manufacturing process according to the present invention has the capacity to remove even more than 12 log of parvovirus B19 (Example 2), complete removal of parvoviruses potentially present in the starting plasma pool is achieved.

The product manufactured by the novel process is, thus, a truly virus-free immunoglobulin product. As apparent to a person skilled in the art, this calculation can be applied to any blood-borne virus, which is not effectively inactivated by chemical inactivation methods, such as hepatitis A virus, and potential so far unknown viruses. Parvovirus is used as an example herein because it is one of the most difficult viruses to completely remove from plasma products.

A further benefit of the current process as compared to prior art is its simplicity. The process can start from fraction II+III paste of human plasma and replace two of the four ethanol fractionation steps of the Cohn-Oncley process. Only one chromatography column is needed to ensure purity of the final product, whereas in the other process described to give high yield of immunoglobulins from fraction II+III paste two chromatography columns are required. The purity of the final immunoglobulin solution is greater than 98%.

Previously, filtration of IVIG products with virus removal filters, which are capable to effectively remove even small viruses such as parvovirus, has been relatively expensive. This was due to the limited amount of IVIG, which could be filtered before the relatively costly filters became clogged. The current invention makes it possible to filter even about 10 kg of IVIG protein with high yield through 1 m² of a virus-removal filter, which means greatly decreased manufacturing costs (Examples 1, 3 and 5).

Another method for polymer removal according to the present invention is treatment with an adsorbent, such as fumed silica (applied, for example, in the form of a colloidal dispersion of silica). Generally, any non-toxic, finely-divided silica-based resin, capable of adsorbing protein aggregates can be used. Treatment of immunoglobulin solution with 0.05-0.5% fumed silica (such as Aerosil 200) effectively removes polymers and improves immunoglobulin throughput in virus filtration, as demonstrated by Example 6. The adsorbent is removed by filtration or centrifugation.

Surprisingly, the immunoglobulin solution can be highly concentrated without precipitation of immunoglobulin. Thus, we have been able to prepare clear solutions containing up to 20 to 25% immunoglobulin. Such solutions provide additional advantages in connection with, e.g., subcutaneous administration of immunoglobulin.

A pharmaceutical composition of IVIG manufactured according to the present invention is suitable for all known clinical uses of IVIG (Knezevic-Maramica and Kruskall, Transfusion 43, 1460-1480, 2003). Because the composition is free from even physico-chemically resistant viruses, such as parvovirus, it is particularly suitable for the treatment of patients susceptible to complications caused by parvovirus, such as pregnant women, immunocompromized patients and patients with underlying hemolytic disorders or otherwise increased erythropoiesis. The daily dose of the IVIG is about 10 mg to 10 g/kg, in particular about 100 mg to 1 g/kg. For hyperimmune globulin, such as anti-D immuno-globulin, and monoclonal antibodies the doses can be lower, such as 10 μg to 10 mg/kg.

The immunoglobulin compositions can be parenterally or enterally administered. The parenteral administration routes include: intravenous, intramuscular, subcutaneous, rectal, intraocular, intrasynovial, transepithelial including transdermal, ophthalmic, sublingual and buccal. In particular, the present immunoglobulin compositions can be formulated for intravenous, subcutaneous or intramuscular administration.

In summary, the manufacturing method according to the present invention has, by far, greater capacity of removing physico-chemically resistant viruses than the manufacturing methods described in prior art. Importantly, the main virus removal steps in the present method, PEG precipitation and virus filtration, are robust and not sensitive to differences in the physico-chemical characteristics of viruses which influence their effective removal e.g. in anion exchange chromatography.

According to a preferred embodiment, the present immunoglobulin solutions are formulated into parenteral compositions by adding trehalose—either as such or in the form of a mixture of trehalose and other conventional stabilizers—to the IgG solution and pH is adjusted if necessary. The solution is then sterile filtered and filled aseptically to final containers, such as vials. These novel pharmaceutical compositions are disclosed in more detail in our copending application titled: “Pharmaceutical Compositions”, the content of which is herewith incorporated by reference.

It should be pointed out that the present invention allows for concentrating the IgG to high concentrations. Generally, the immunoglobulin is recovered in the form of a liquid composition having a concentration of 1 to 250 g/l, although the suitable concentration of polyclonal immunoglobulin for its conventional therapeutic uses is in the range of some 10 to 250 g/l, e.g. 50 to 200 g/l. The present invention makes it possible to produce such highly concentrated polyclonal IVIG compositions, which allows for facile administration of large amounts of immunoglobulins not only intraveneously but also subcutaneously and intramuscularly. One particular advantage of subcutaneous administration is that it for home-treatment of patients with high doses of immuno-globulins.

The following non-limiting examples illustrate the invention.

In the examples, the following analytical procedures were followed:

IgG was determined by immunoturbidimetry with a kit from ThermoClinical Labsystems;

IgA was determined by ELISA (Hirvonen et al., J Immunol Methods 163, 59-65, 1993); and IgG subclasses by ELISA (PeliClass ELISA kit, Sanguin). Purity was determined by zone electrophoresis on agarose and molecular size distribution by size-exclusion liquid chromatography according to Ph. Eur. 3rd Ed. 1997:0338. Caprylic acid was determined by gas chromatography according to Ph. Eur. 2001:1401 and and polyethylene glycol as described by Skoog (Vox Sang 37, 345-349, 1979).

### EXAMPLE 1

This example describes manufacturing of aggregate-free and virus-safe immunoglobulin from human plasma with high yield.

Fraction II+III paste from human plasma was fractionated by the Cohn method (Krijnen, Chemie en Techniek 25,193-196, 1970). 500 g of fraction II+III paste was suspended in 8 volumes of purified water at about 5° C. and the pH was adjusted to 4.8 with 0.2 mol/l acetic acid. The suspension was brought to room temperature (about 22° C.). Caprylic acid was added to a concentration of 50 mM during 1 hour. The suspension was mixed for I hour and the precipitate was removed by centrifugation. The pH of the solution was raised from 4.5 to 5.4 with 0.2 M NaOH, 30 g/l of PEG 4000 was added and the solution was mixed for 16 hours. 2% of diatomaceous earth was added and the mixture was filtered. The solution conductivity was adjusted to 2.0 mS/cm using sodium acetate buffer. The filtrate was applied to a column of ANX Sepharose FF gel equilibrated with 20 mM sodium acetate buffer, pH 5.4. The flow through fraction containing IgG was recovered. The pH of the solution was adjusted to pH 4.4 using 0.5 M acetic acid. After filtration through a 0.1 μm prefilter, the solution was filtered through a Viresolve NFP virus filter at 35° C. with a pressure of 3.5 bar. Protein concentration was about 8 g/l and a load of about 10 kg IgG/m² filter area was used. The solution was filtered in about 6 hours with an IgG yield of about 98-100%. The solution was concentrated by ultrafiltration, diafiltered with water for injection to remove polyethylene glycol, and finally concentrated. The concentrated solution was formulated to a 10% IgG solution, pH to 4.4, containing 0.2 M glycine. Another 15% IgG formulation, pH to 5.2, containing 0.2 M trehalose was also prepared. The formulated solutions were sterile filtered into final containers.

The overall yield from dissolved paste to final product was about 64% (see the Table 1). This is 30-70% higher than obtained with conventional processes based on cold alcohol fractionation. Most of the aggregates were removed in the PEG precipitation step and no aggregates or polymers were present in the final product.

TABLE 1
IgG yield and occurrence of aggregates after the different process steps.

| | IgG yield | | Aggregates |
|---|---|---|---|
| Process step | g/kg plasma | % | % |
| Suspended II + III paste | 7.5 | | 4 |
| After caprylic acid treatment | 6.8 | 91 | 5 |
| After PEG precipitation | 5.7 | 76 | 0.1 |
| After chromatography | 5.1 | 68 | 0.0 |
| After virus filtration | 5.0 | 67 | 0.0 |
| Final product | 4.8 | 64 | 0.0 |

### EXAMPLE 2

This example demonstrates reduction of parvovirus B19 in the manufacturing process described in Example 1.

Virus reduction in each process step was studied by spiking the starting solution with high-titer parvovirus B19 positive plasma. Nucleic acids were isolated from the starting solution and processed samples diluted in parvovirus-negative plasma with the Roche MagNA Pure method. The amount of parvovirus B19 DNA was determined by real-time PCR using the Roche Lightcycler and the Roche Parvovirus B19 Quantitation Kit. The reduction of parvovirus in the different process steps is shown in Table 2.

TABLE 2
Reduction of parvovirus in the process steps.

| | Reduction factor |
|---|---|
| Process step | log 10¹⁰ |
| Caprylic acid precipitation | 1.7 |
| PEG precipitation | 4.8 |
| ANX chromatography | 2.0 |
| Virus filtration | 4.1 |
| Total reduction factor | 12.6 |

### EXAMPLE 3

This example demonstrates the importance of a specific polymer removal step for efficacious virus filtration. A crude immunoglobulin solution was prepared and treated with caprylic acid as described in Example 1. The pH of the supernatant solution was raised to 5.4. In separate experimental batches different amounts of polyethylene glycol (PEG 4000) or no PEG was added to the supernatant solution. The solution was mixed at room temperature for 16 hours, 2% diatomaceous earth was added and the solution was clarified by filtration. The clarified solution was subjected to anion exchange chromatography on ANX Sepharose and the effluent containing purified IgG was recovered. The pH of the effluent solution was adjusted to 4.4. After prefiltration with a 0.1 μm filter, the solution was filtered with Viresolve NFP (Millipore) filter with a pressure of 3.5 bar at 35° C. Protein concentration was about 8 g/l and a load of about 10 kg IgG/m² filter area was used. Filtrate flux was monitored by recording filtrate weight. PEG treatment remarkably improved both the filtrate flux and IgG throughput (Table 3).

TABLE 3
Influence of PEG treatment on filtrate flow and IgG throughput in virus
filtration.

| | Initial filtrate | IgG throughput (kg/m²) | | |
|---|---|---|---|---|
| | flux | Flux 80% | Flux 50% | Flux 30% |
| PEG concentration | (kg/m²/h) | of initial | of initial | of initial |
| No PEG | 42 | <1 | <1 | <1 |
| 2% PEG | 141 | 2.8 | 5.0 | 6.1 |
| 3% PEG* | 223 | 13.2 | 18.8 | n.a. |

*Mean values from two separate experiments;
n.a. not analyzed.

### EXAMPLE 4

This example demonstrates the importance of pH of the immunoglobulin solution on efficacious virus filtration. Aggregate-free immunoglobulin solution was prepared from Cohn fraction II+III of human plasma by caprylic acid treatment, PEG precipitation and ion exchange chromatography as described in Example 1. The pH of the effluent solution was adjusted to different values from 4.2 to 5.2. After prefiltration with a 0.1 Jim filter, the solutions were filtered with Viresolve NFP filters with a pressure of 3.5 bar. Temperature was 23° C. for the pH 5.2 solution and 35° C. for the two other solutions. The highest initial flux and IgG throughput was obtained when pH of the effluent solution was adjusted to 4.4 (Table 4).

TABLE 4
Effect of pH of the immunoglobulin solution on filtrate flow and IgG
throughput in virus filtration.

| | Initial filtrate | IgG throughput (kg/m²) | | |
|---|---|---|---|---|
| | flux | Flux 80% | Flux 50% | Flux 30% |
| pH of solution | (kg/m²/h) | of initial | of initial | of initial |
| 5.2 | 192 | <1 | n.a. | n.a. |
| 4.4* | 223 | 13.2 | 18.8 | n.a. |
| 4.2* | 164 | 3.6 | 6.1 | 7.3 |

*Mean values from two separate manufacturing batches are shown.

### EXAMPLE 5

This example demonstrates the high efficacy obtained in virus filtration of immunoglobulin solution manufactured according to the present invention as compared to pepsin treatment.

Aggregate-free IgG solution was prepared from Cohn fraction II+III of human plasma by caprylic acid treatment, PEG precipitation and ion exchange chromatography as described in Example 1. Another pure IgG solution was prepared from Cohn fraction II of human plasma by DEAE-Sephadex treatment, ultrafiltration and pepsin treatment according to Published International Patent Application WO 96/35710. After prefiltration with a 0.1 μm filter, the solutions were filtered with a Viresolve NFP filter with a pressure of 3.5 bar at 35° C. Protein concentration was about 8 g/l. Much higher IgG throughput was obtained when filtering the solution manufactured by the new process than the solution manufactured by pepsin treatment (Table 5).

TABLE 5
Effect of manufacturing process on virus filtration of a pure
immunoglobulin solution.

| Immunoglobulin | Initial filtrate | IgG throughput (kg/m²) | | |
|---|---|---|---|---|
| manufacturing | flux | Flux 80% | Flux 50% | Flux 30% |
| process | (kg/m²/h) | of initial | of initial | of initial |
| New process* | 223 | 13.2 | 18.8 | n.a. |
| Pepsin treatment | 142 | 0.8 | 1.9 | 2.9 |

*Mean values from two separate manufacturing batches are shown.

### EXAMPLE 6

This example demonstrates removal of polymers and improved filtration efficacy of immunoglobulin solution achieved by fumed silica treatment. In a first set of experiments, a crude immunoglobulin solution was treated with caprylic acid as described in Example 1 and pH of the supernatant solution was raised to 5.4. To one portion of the solution, 0.2% of fumed silica (Aerosil 200) was added and the suspension was mixed for one hour. 2% diatomaceous earth was added and the suspension was filtered. Another portion of the supernatant solution was subjected to clarification filtration with 2% diatomaceous earth only. Both solutions were subjected to anion exchange chromatography on ANX Sepharose and the effluent containing purified IgG was recovered. The fumed silica-treated immunoglobulin solution did not contain any detectable polymers, whereas the reference solution contained 0.4% polymers.

In another set of experiments a pure IgG solution obtained from solubilized Cohn fraction II of human plasma by DEAE-Sephadex treatment and ultrafiltration according to WO 96/35710. Protein concentration was adjusted to 10 g/l. One portion of the solution was treated with 0.2% Aerosil, 2% diatomaceous earth was added and the suspension was filtered. Another portion of the solution was treated with pepsin according to WO 96/35710. A third portion served as a reference. After prefiltration with a 0.1 μm filter, the solutions were filtered with Viresolve NFP filters with a pressure of 3.5 bar at 35° C. IgG throughput was about 50% higher after Aerosil-treatment than pepsin treatment. Both treatments increased IgG throughput several fold as compared to the reference solution.

1. A process for preparing a purified, essentially virus-safe immunoglobulin preparation, said process comprising the steps of
a) subjecting a starting solution comprising immunoglobulin and polymeric proteins to at least one virus-inactivation step, in which the composition is contacted with caprylic acid to form a precipitate and a supernatant solution comprising dissolved immunoglobulin and polymeric proteins,
b) recovering the supernatant solution,
c) contacting the supernatant solution with at least one ion exchange resin to produce a first effluent comprising immunoglobulin,
d) recovering the first effluent,
e) subjecting the first effluent to nanofiltration on a filter having an average pore size of about 10 to 40 nm to remove any enveloped and non-enveloped viruses and to produce a second effluent,
f) recovering the second effluent, and
g) formulating it to a pharmaceutically acceptable, virus-safe immunoglobulin preparation, which is free from polymeric proteins,
wherein polymeric proteins are removed from the supernatant solution obtained from step b by adding polyethylene glycol to the supernatant solution.

2. The process according to claim 1, wherein step a is carried out by adding caprylic acid to a final concentration of 15-60 mmol/l, preferably to 20-50 mmol/l.caprylic acid.

3. The process according to claim 2, wherein step a is carried out at a pH of about 4.0 to 5.0.

4. The process according to claim 1, wherein the starting solution is provided by dissolving an immunoglobulin-containing blood fraction in an aqueous solution at a pH of about 4.0 to 5.0, preferably at 4.5 to 5.0.

5. The process according to claim 1, wherein the pH of the supernatant solution of step b is adjusted to a value of about 5.3 or higher.

6. The process according to claim 1, wherein the concentration of the polyethylene glycol is 2 to 4% by weight of solution.

7. The process according to claim 1, wherein the supernatant solution contains caprylic acid in a concentration of about 1 to 20 mmol/l.

8. The process according to claim 1, wherein step e is carried out at a pH of 4.2 to 5.0.

9. The process according to claim 1, wherein the starting plasma contains less than 10⁴ IU/ml of parvovirus B19 DNA.

10. The process according to claim 1, wherein the starting plasma is obtained from Cohn fraction II+III paste of human plasma.

11. A method of efficaciously filtering immunoglobulin solutions on a nanofilter having a pore size of 10 to 40 nm, which comprises conducting through the filter an immunoglobulin solution, comprising 1 to 25 g/l immunoglobulin, wherein the filtration is carried out at a pH of about 4.2 to 5.0 and wherein the immunoglobulin solution further contains no detectable polymer aggregates, to remove at least 3 log of viruses with particle size of about 20 nm, said immunoglobulin solution being obtained from a crude immunoglobulin solution by
subjecting the crude immunoglobulin solution to caprylic acid treatment,
removing protein aggregates and viruses from the immunoglobulin solution by adding polyethylene glycol, and
subjecting the immunoglobulin solution to anion exchange chromatography in order to purify the crude immunoglobulin solution and to produce a solution, which is free from detectable amounts of protein aggregates.

12. The method according to claim 11, wherein the immunoglobulin solution contains 2 to 4 wt-% polyethylene glycol.

13. The method according to claim 11, wherein the solution is filtered at a temperature of about 20 to 50° C. and at a pressure difference of about 0.2 to 8 bar.

14. The method according to claim 13, wherein the solution is filtered using a trans-membrane pressure of 0.5 to 5.5 bar.

15. The method according to claim 11, wherein at least 5 kg, preferably at least 7.5 kg, of immunoglobulin is passed through 1 m² of filter area with less than 50% decrease in filter flux.

16. The method according to claim 11, wherein the immunoglobulin solution is filtered on a composite virus-removal filter.

17. The method according to claim 11, wherein filtration is carried out at a pH of about 4.2 to 4.8.
